(19) United States
(12) Patent Application Publication
(10) Pub. No.: US 2017/0151347 A1
(43) Pub. Date: Jun. 1, 2017

(54) FUNCTIONALIZED NANOPARTICLES WITH ENCAPSULATED CARGO AND METHOD OF THEIR SELF-ASSEMBLY

(71) Applicants: Berney PENG, Arlington, MA (US); Igor SOKOLOV, Medford, MA (US)

(72) Inventors: Berney PENG, Arlington, MA (US); Igor SOKOLOV, Medford, MA (US)

(21) Appl. No.: 15/363,077

(22) Filed: Nov. 29, 2016

Related U.S. Application Data
(60) Provisional application No. 62/260,569, filed on Nov. 29, 2015.

(51) Int. Cl. A61K 41/00; A61K 9/14

(52) U.S. Cl. CPC A61K 47/48961; A61K 41/0057; A61K 47/4823; A61K 47/48923; A61K 9/146

(57) ABSTRACT

This application discloses the approach of synthesizing cellulose acetate nanoparticles and rods which may have a chemically functionalized surface and an encapsulated cargo load. Functionalization and/or loading of the cargo are made through a physical mixing of the functionalizing and/or cargo components in the synthesizing bath. This can result in particles with functionalized surfaces with various functional groups, as well as active cargo load encapsulated in the particles. The encapsulated cargo includes but is not limited to biologically, chemically, and optically active substances.

### CROSS-REFERENCE TO RELATED APPLICATIONS

The present patent application claims priority to U.S. Provisional Patent Application No. 62/260,569, filed on Nov. 29, 2015.

### FIELD OF THE INVENTION

The invention relates to the synthesis of cellulose acetate nanoparticles and rods which have various functionalities and encapsulated cargo.

### BACKGROUND FOR THE INVENTION

The first reported synthesis of cellulose acetate (CA) nanoparticles was performed in 2008. The ability of composite nanoparticles to encapsulate hydrophobic substances within aqueous media or by further surface functionalization possess potential utility in pharmaceutical and bio- or food technology. Beyond cellulose acetate, other polysaccharide nanoparticles including cellulose variants have been demonstrated for delivery and imaging; each approach retains specific strengths, weaknesses, forms, and applications.

### SUMMARY OF THE INVENTION

There is a need for the following embodiments of the invention. Of course, the invention is not limited to these embodiments. This invention describes the synthesis of cellulose acetate nanoparticles having diameters ranging from 30-200 nm, and rods with diameters of 50 nm-10 microns, and an aspect ratio 100:1 and above. We further disclose the use of the same in application in which the chosen functionality and cargo load can be useful. Nonrestrictive examples include development of bright fluorescent nanoparticles and rods which could be used for imaging. While CA is relatively hydrophobic overall, the ability of CA to assemble into stable, nanoscale particles via precipitation techniques reflects amphiphilic functionality along the backbone. This heterogeneity enables CA to interface with numerous polymers beyond the functionalized, surface adsorbed polysaccharides. Overall, we disclose the assembly of composite, surface-functionalized CA nanoparticles. The following brief details particle morphological control, quantifies (physical) encapsulation range and extent, and evaluates non-covalent functionalization with multiple polymer co-assemblies.

According to an embodiment of the invention, a process comprises:
(a) morphology control of the nanoparticles and rods through varying the solvent surface tension and polarity during nanoprecipitation synthesis; polymer dispersion quality controlled by dissolved polymer concentration and polymer-solvent affinity; rate of introducing dissolved polymer and potential guests with strong agitation;
(b) cargo loading; the disclosed is the ability of cellulose acetate to physically encapsulate molecular and polymeric guests of varying hydrophobicity;
(c) functionalization of the particle surface; we disclose a non-covalent functionalization of cellulose acetate nanoparticles and rods by physical association during the precipitation process; the functional molecules are physically added to the synthesizing bath during the synthesis.

The present invention, as disclosed, comprises both a particle and the method of making the particle.

In particular, the present invention comprises a particle, comprising cellulose acetate and a cargo agent, wherein the cargo agent is non-covalently bonded to the cellulose acetate.

In some aspects, the cargo agent is fluorescent. In some aspects, the particle exhibits fluorescent ultrabrightness. In some aspects, the particle exhibits fluorescence in the near infrared part of the spectrum. In some aspects, the cargo agent is IR813, IR143, Indocyanine Green (ICG), Methylene Blue, or a combination thereof.

In some aspects, the cargo agent is a hydrophobic drug. In some aspects, the cargo agent is Camptothecin or Doxyrubicin.

In some aspects, the particle further comprises a general polymer that is physically bonded to the cellulose acetate. In some aspects, the general polymer is present on the particle surface, thereby functionalizing the particle surface. In some aspects, the general polymer is either an ionic surfactant, a non-ionic surfactant, or a charged polyelectrolyte. In some aspects, the general polymer is Polyvinylpyrrolidone (PVP), Pluronic-F 127 (PF127), Polyethyleneimine (PEI), or polyethylene glycol (PEG). In some aspects, the particle has a surface chemistry, said surface chemistry being defined by a presence of surface hydroxyl groups.

In some aspects, the cargo agent dictates a core crystallinity of the particle.

In some aspects, the particle has a spherical shape. In some aspects, the particle has a rod shape. In some aspects, the particle has a size of 30-500 nm. In some aspects, the particle has a shape, the shape being a sheet or a branch.

Also disclosed is a method for making the particle, comprising dissolving a cellulose acetate in an organic solvent, dissolving a molecular guest in said organic solvent, adding said cellulose acetate and said molecular guest in said organic solvent to a miscible non-solvent, said adding being a dropwise adding coupled with stirring, removing said solvent, and precipitating one or more particles, each particle comprising said cellulose acetate and said molecular guest, wherein said precipitating forms non-covalent bonds between said cellulose acetate and said molecular guest.

In some aspects, the solvent is removed via vacuum. In some aspects, the solvent is removed via dialysis.

In some aspects, the molecular guest is a fluorescent dye.

In some aspects, the organic solvent possesses a polarity index between 4 and 7.5.

In some aspects, a nature of the organic solvent determines at least one of; particle size, particle surface chemistry, and particle core crystallinity.

In some aspects, a nature of the molecular guest determines at least one of: particle size, particle shape, particle surface chemistry, and particle core crystallinity.

In some aspects, the method further comprises the step of adding a general polymer. In some aspects, the general polymer is present on the particle's surface, thereby functionalizing the particle. In some aspects, the general polymer is an ionic surfactant, a non-ionic surfactant, or a charged polyelectrolyte. In some aspects, the general polymer is Polyvinylpyrrolidone (PVP), Pluronic-F 127 (PF127), Polyethyleneimine (PEI), polyethylene glycol (PEG), or a combination thereof. In some aspects, the general polymer determines at least one of: particle size, particle surface chemistry, and particle core crystallinity.

### BRIEF DESCRIPTION OF DRAWINGS

FIG. 1. CA particle effective sizes shown as a function of polymer concentration and solvent polarity. Ideal conditions minimizing diameters are 1-2 mg/ml polymer concentration, maximizing dispersion, and a solvent polarity index from 4-5.5.

FIG. 2. Dissolved polymer in organic solvent is added dropwise with agitation to a miscible non-solvent in Step 1. As organic solvent is displaced, the cellulose will assemble into spherical particles (SEM picture) to minimize interfacial energy with the non-solvent. Solvent, generally acetone or tetrahydrofuran, is removed by vacuum drying and/for dialysis in Step 2. In the Figure, A stands for DHF, B for acetone, and C for DMSO.

FIGS. 3A and 3B show SEM of cellulose acetate rods with 20:1 aspect ratio. FIG. 3A demonstrates the diversity in needle morphology from a single assembly. FIG. 3B illustrates a bundle of smaller, more homogeneously sized needles.

FIG. 4A shows a cross-polarized microscope image of the end of a cellulose acetate microneedle assembled at a bulk scale. The total needle length is several hundred microns. FIG. 4B is an Atomic Force Microscopy (AFM) image showing the representative topology of a 300 nm-wide needle (top).

FIG. 5. Normalized intensity spectra of encapsulated R6G versus free R6G. The spectrum of encapsulated R6G appears slightly shifted (4 nm) and slightly broader. Excitation performed at 488 nm.

FIG. 6. Nile Red spectrum. Excitation performed at 550 nm.

FIG. 7. Graph of incapsulated versus free dye photostability, illustrating greater normalized integrated fluorescence over time of CA particles with encapsulated dye.

### DESCRIPTION OF PREFERRED EMBODIMENTS

Definitions.

It should be noted that the term “physical encapsulation” is synonymous with “non-covalent encapsulation,” as the terms are both used herein.

The fluorescent brightness of a fluorescent particle is referred to as “fluorescent ultrabrightness” (or simply, “ultra-bright”) when the brightness of the particle is higher than the maximum fluorescent brightness coming from a particle of the same size and comprising quantum dots of a similar spectrum encapsulated in a polymer matrix.

Reference in this specification to “one embodiment,” “an embodiment,” “one version,” “a version,” should be understood to mean that a particular feature, structure, or characteristic described in connection with the version, or embodiment is included in at least one such version, or embodiment of the disclosure, and may be included in more than one embodiment or version. The appearances of phrases “in one embodiment”, “in one version,” and the like in various places in the specification are not necessarily all referring to the same version, or embodiment, nor are separate or alternative versions, variants or embodiments mutually exclusive of other versions, variants, or embodiments. Moreover, various features are described which may be exhibited by some versions, or embodiments and not by others. Similarly, various requirements are described which may be requirements for some versions, variants, or embodiments but not others. If the specification states a component or feature “may”, “can”, “could”, or “might” be included or have a characteristic, that particular component or feature is not required to be included or have the characteristic.

Furthermore, as used throughout this specification, the terms ‘a’, ‘an’, ‘at least’ do not denote a limitation of quantity, but rather denote the presence of at least one of the referenced item, and any usage of the term ‘a plurality’ denotes the presence of more than one referenced items.

Morphology Control

Nanoparticles

Cellulose acetate nanoparticle morphologies are broadly manipulated by controlling initial polymer dispersity and the interface between polymer/solvent and miscible non-solvent. Polymer affinity, polarity, and surface tension are all physical properties of organic solvents that directly influence particle size and polydispersity. Gibbs-Marangoni effect and supersaturation are regarded as relatively comprehensive models of nanoprecipitation with polymeric materials. While these theories are consistent with most synthesis, we have observed assembly inconsistencies affecting control depending on direction of nanoprecipitation interface and polymer-solvent similarity. For example, the addition of dissolved polymer in acetone added to hexane versus water produces significantly different sizes despite similar polarity differences (290 nm vs. 60 nm effective diameter, respectively).

In the present disclosure, CA nanoparticle size ranges from 30-200 nm (number average) and 60-300 nm (effective diameter). Utilizing a dropwise, slow addition of dissolved polymer solution process, polydispersity (PDI) spans 0.10 to 0.24 without molecular or polymer guest encapsulation and without post-synthesis filtering. The following charts illustrate the impact of solvent physical properties—specifically polarity and surface tension—on CA particle size. Control is exhibited across polymer concentration, polarities index, and surface tension.

TABLE 1

| Solvent | Surface Tension (mN/m) |
|---|---|
| Hexane | 18.43 |
| Acetone | 25.20 |
| THF | 26.40 |
| DMSO | 43.54 |
| Water | 72.80 |

Table 1 shows the range of surface tensions suggesting direction of interfacial movement during precipitation.

### EXAMPLE 1

### Synthesis of CA Nanoparticles

Cellulose acetate nanoparticles are prepared by nanoprecipitation. 1 or 2 mg/ml of 50,000 MW cellulose acetate is dissolved in solvent (preferably, tetrahydrofuran or acetone) and added drop wise into the miscible non-solvent (preferably, water) at a 5:1 water to organic ratio (preferable range from 3:1 to 6:1 ratios). Polymer solution addition rates span preferably from 1 ml/min to 5 ml/min, and are done so under vigorous stirring. Solvent is removed either under vacuum overnight or by dialysis. For dialysis, a cellulose membrane (Spectra/Por) with 12-14 KD cutoff is preferable. Particles are dialyzed according to standard practices.

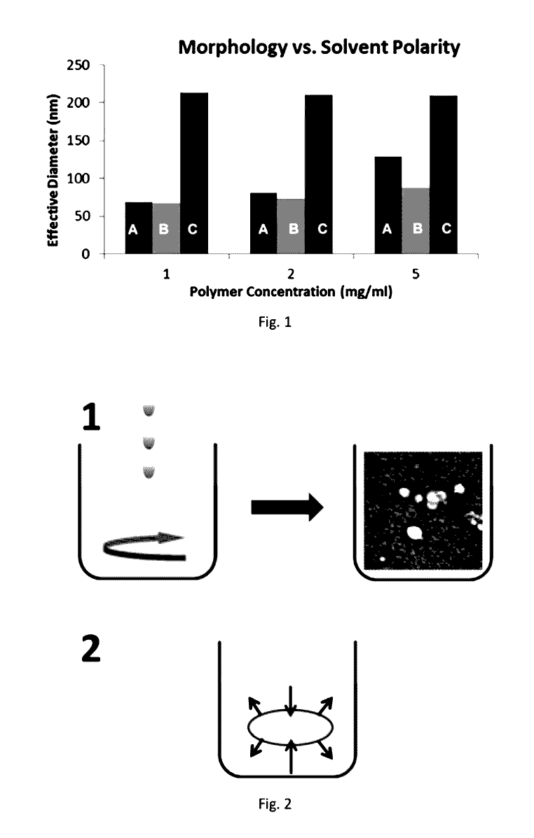

FIG. 2 summarizes the process via illustration: dissolved polymer in organic solvent is added dropwise with agitation to a miscible non-solvent in Step 1. As organic solvent is displaced, the cellulose acetate assembles into spherical particles (SEM picture). Preferable organic solvents for usage possess polarity indexes between 4-7.5 and are removed by vacuum drying and/or dialysis in Step 2.

Rods

We disclose that a dye of a family of conjugated hydrophobic dyes, exampled by Nile Red dye produces rod-shaped particles possessing high aspect ratios of tunable dimension (for example, 100:1 and above). Examples of assembled rods features as small diameter as 50 nm and 1-2 microns in length, or as large as several microns in diameter and several hundred microns in length. This invention represents the first case of this type of assembly (i.e. physical encapsulation creating non-covalent bonding) using cellulose acetate or even cellulose variants. It should be noted that the process of physical encapsulation as described herein leads to a specific product of non-covalently bonded cellulose acetate (or other variants) and a contrast agent (e.g. fluorescent dye). Thus, the product and process of synthesizing the product are one and the same. A different process would lead to a different structure than that claimed herein below.

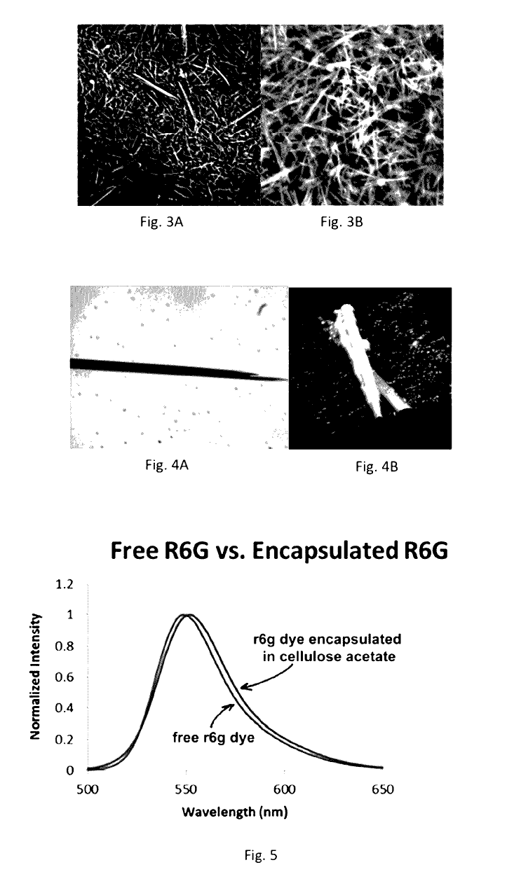

FIG. 3 shows Scanning Electron Micrographs (SEM) of cellulose acetate-Nile Red composite, rod-shape particles. The rods range from less than 50 nm in diameter (FIG. 3B) to 200-300 nm (FIG. 3A). Lengths span from 1 to 8 μm. FIG. 4A represents a cross-polarized image of a cellulose acetate rods grown in bulk. These rods are several hundred microns in length and possesses a smooth, transparent surface. FIG. 4B examples an Atomic Force Microscopy (AFM) image of two rods laying across one another.

FIG. 3 shows an SEM of cellulose acetate rods with 20:1 aspect ratio. FIG. 3A demonstrates the diversity in “needle” morphology from a single assembly. FIG. 3B illustrates a bundle of smaller, more homogeneously sized needles.

FIG. 4A shows a cross-polarized microscope image of the end of a cellulose acetate microneedle assembled at a bulk scale. The rod lengths are several hundred microns. FIG. 4B is an AFM image showing the representative topology of a 300 nm wide rod-like particle (top). It appears the rod possesses a folded or tube-like structure, and while the top rod is tapered, the bottom particle has a flatter topography.

### EXAMPLE 2

### Synthesis of CA Rods

1 or 2 mg/ml of 50,000 MW cellulose acetate is dissolved in an organic solvent capable of dissolving cellulose acetate along with Nile Red dye. The mixture is added drop wise (preferably 1-5 ml/min) into water at a preferable ratio of 5:1 water to organic under stirring. The solution turns purple together with the formation of nanoparticles. The rod assemblies begin to grow after about 30 seconds to 2 minutes post-precipitation. Solvent is removed either under vacuum or by dialysis. For dialysis, a cellulose membrane (Spectra/Por) with 12-14 KD cutoff is used. Particles are dialyzed using standard practices. Due to the small solubility of Nile Red in water, removal of solvent can cause free Nile Red to precipitate out of solution. These precipitates can be filtered out using standard filtration, for example, with filter paper of preferable pore size of 5 microns or larger.

Encapsulation of Cargo

Here we disclose the ability of cellulose acetate to physically encapsulate molecular and polymeric guests of varying hydrophobicity/hydrophilicity. The type of guest defines future usage of the particles. For example, guests may extend to therapeutic drug delivery applications. Numerous chemotherapy agents such as Camptothecin and Doxyrubicin are quite hydrophobic in nature and necessitate a particle possessing a hydrophobic core exhibiting stability in aqueous environments.

Here, we provide, as an example, the encapsulation efficiency of guest molecules using fluorescent dyes as guests. Molecular guests of all types have been encapsulated including hydrophobic and hydrophilic IR dyes such as IR813, IR143, Indocyanine Green (ICG), Methylene Blue, and others CA nanoparticles exhibit a degree of capturing anywhere from 100 to 350 dye molecules per normalized 40 nm diameter particle based on absorbance and fluorescence measurements. Dyes remain associated well enough with the particle architecture such that high fluorescent signal remains despite diminishment in quantum yield. Further, the ability to capture such a large quantity of dye could be useful in photodynamic therapy (PDT). Table 2 (below) depicts the encapsulation ability of CA nanoparticles with an FDA-approved IR dye, Indocyanine Green (IR125 or ICG). The number of dye molecules encapsulated per 40 nm diameter particle is assessed by comparing fluorescent intensity of particles versus free ICG in water solution. One can note that the effective amount of dye molecules encapsulated allows one to speak about fluorescent ultrabrightness (i.e., brighter than particles of similar fluorescent spectra assembled with quantum dots or just quantum dots).

TABLE 2

| Particle Type* | # Dye Molecules** | Z-ave (nm) | PDI |
|---|---|---|---|
| 10E−6 M IR125/TEA | 27 | 84 | 0.23 |
| 10E−2 M IR125/TEA | 353 | 144 | 0.15 |

*2 mg/ml Cellulose
**Normalized to 40 nm diameter particle

Table 2 demonstrates CA nanoparticle encapsulation ability using FDA-approved IR125. It appears that high encapsulation influences morphology. Generally, encapsulated particles range from 50 to 150 nm number-based average (80-300 nm in effective diameter).

FIG. 5 shows spectra of encapsulated Rhodamine 6G (R6G) dye compared with free R6G in water. The fluorescent properties of R6G encapsulated CA particles demonstrate enough hydrophilic interaction between the core of the particle and dye to minimize quenching.

As described in an earlier section on CA rod assembly, Nile Red dye, a lipophilic dye with low solubility in water, exhibits a strong fluorescence when encapsulated. While CA can be used to encapsulate hydrophilic dyes, it is ideally suited for hydrophobic guest encapsulation. FIG. 6 shows Nile Red fluorescent spectrum excited with light of 550 nm wavelength.

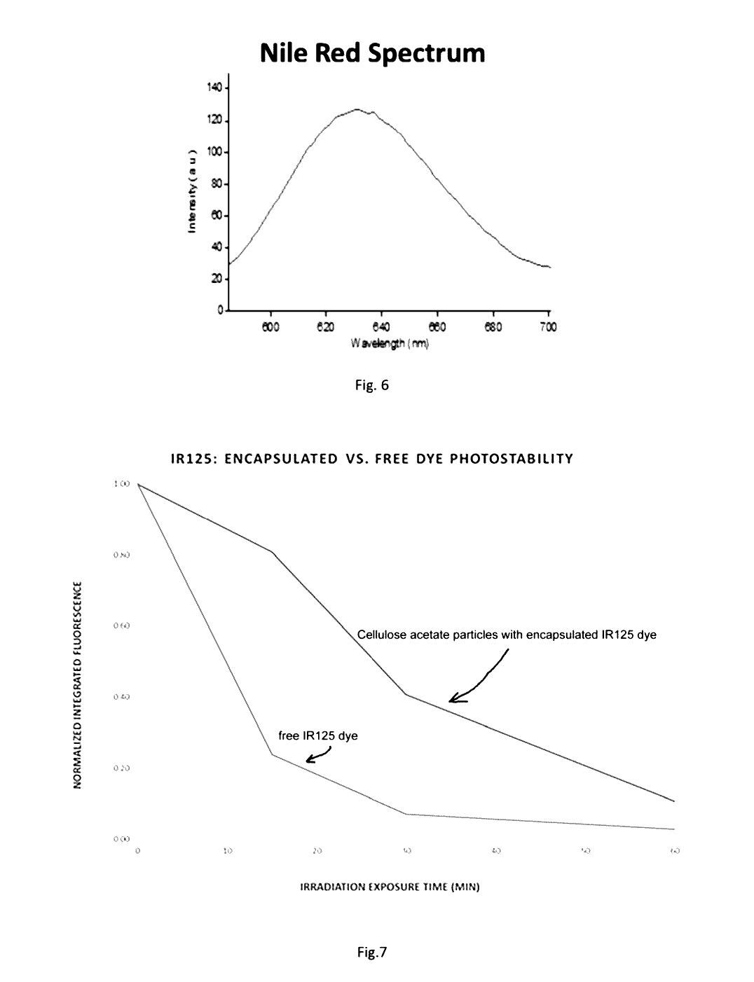

An additional important property of the fluorescence of the cellulose acetate particles, as described herein, is an excellent photostability, or resistance to photobleaching compared to pure dye. FIG. 7 demonstrates an example of encapsulation of near infrared fluorescent dye, IR125, in a cellulose acetate nanoparticle matrix. Increased photostability not only implies successful physical encapsulation, but also suggests the dye-cellulose interaction is specific and significant enough to impart superior stability versus non-encapsulated dye.

### EXAMPLE 3

### Synthesis of Fluorescent CA Nanoparticles

Cellulose acetate is dissolved with the fluorescent dye in an organic solvent capable of dissolving cellulose acetate prior to precipitation. If the guest is insoluble in organic solvent, a mixed solvent mixture, an emulsifier, or a hydrophobizing counter-ion is utilized. 1 or 2 mg/ml of 50,000 MW cellulose acetate and a molecular guest is dissolved in organic solvent and added drop wise into an aqueous solution at a 5:1 water to organic ratio, under stirring (in some aspects, the stirring may be vigorous). Solvent is removed either under vacuum overnight or by dialysis. For dialysis, a cellulose membrane (Spectra/Por) with 12-14 KD cutoff is preferable. Particles are dialyzed until fluorescence is undetectable in the dialysate.

Functionalization

It is common in polymeric nanoparticles to conjugate drugs, contrast agents, and biostability enhancing molecules/polymers, like Polyethylene Glycol (PEG), chemically to the backbone prior to assembly. Here, we disclose a non-covalent functionalization of cellulose acetate particles by physical association during the precipitation process.

A range of polymers including ionic and non-ionic surfactants and charged polyelectrolytes (preferably, Polyvinylpyrrolidone (PVP), Pluronic-F 127 (PF127), and Polyethyleneimine (PEI) were successfully co-assembled with cellulose acetate to produce complex particles possessing specific surface chemistries as reflected by zeta-potential measurements post-dialysis. For instance, branched 10K MW PEI, a cationic polymer containing primary, secondary, and tertiary amines, electrostatically anchors into the negatively charged CA particle, producing a positively charged shell. This particle architecture retains a hydrophobic core while exposing reactive primary amines to the aqueous environment. In addition to enhancing stability, the potential of this approach for further surface modification is apparent to those skilled in the art. Addition of reactive NHS-PEG ester effectively conjugates to the surface, PEGylating particles and improving stability and suitability of such particles for in-vivo applications. Table 3 (below) illustrates composite CA particle structure and surface chemistry by examining effective size, polydispersity index, and zeta potential.

TABLE 3
The data showing the effect of polymeric guest on the particle structure
and the presence of the guest on the particle surface.

| Particle Type* | Z-ave (nm) | PDI | Zeta (mV) |
|---|---|---|---|
| Cellulose Acetate (2 mg/ml THF) | 81 | 0.19 | −28.1 |
| Cellulose-Polyvinylpyrrolidone (PVP) | 121 | 0.18 | −8.9 |
| Cellulose-Polyethyleneimine (PEI) | 119.5 | 0.15 | 47.8 |
| Cellulose-Polyethyleneimine/PEG | 116 | 0.14 | 26.0 |
| Cellulose-PF127 | 103 | 0.22 | −11.5 |

*2 mg/ml Cellulose, 1:1 Cellulose to Polymer Ratio except PEI

### EXAMPLE 4

### Functionalized CA Particles

1-2 mg/ml of 50,000 MW cellulose acetate is dissolved in an organic solvent along with an equal concentration (preferably 1:1) of a co-polymer. This mixture is added drop wise into an aqueous solution at a 5:1 ratio of aqueous to organic under vigorous stirring. If the polymeric guest used for surface functionalization is insoluble or poorly soluble in organic solvent, a mixed solvent mixture or emulsifier can be utilized. Solvent is removed either under vacuum overnight or by dialysis. For dialysis, a cellulose membrane (Spectra/Por) with 12-14 KD cutoff was used. Particles are dialyzed according to standard protocols.

The descriptions given here, while indicating various embodiments of the invention and numerous specific details thereof, are given by way of illustration and not of limitation. Many substitutions, modifications, additions and/or rearrangements may be made within the scope of an embodiment of the invention without departing from the spirit thereof, and embodiments of the invention include all such substitutions, modifications, additions and/or rearrangements.

Finally, it should be noted that the term “comprising” does not exclude other elements or features and the “a” or “an” does not exclude a plurality. The verb ‘comprise’ and its conjugations do not exclude the presence of elements or steps other than those listed in any claim or the specification as a whole. The singular reference of an element does not exclude the plural reference of such elements and vice-versa. The mere fact that certain measures are recited in mutually different dependent claims does not indicate that a combination of these measures cannot be used to advantage. Furthermore, elements described in association with different embodiments may be combined. Finally, it should be noted that the above-mentioned examples, and embodiments illustrate rather than limit the invention, and that those skilled in the art will be capable of designing many alternative embodiments without departing from the scope of the invention as defined by the appended claims. As equivalent elements may be substituted for elements employed in claimed invention to obtain substantially the same results in substantially the same way, the scope of the present invention is defined by the appended claims, including known equivalents and unforeseeable equivalents at the time of filing of this application. Thus, in closing, it should be noted that the invention is not limited to the abovementioned versions and exemplary working examples. Further developments, modifications and combinations are also within the scope of the appended patent claims and are placed in the possession of the person skilled in the art from the present disclosure. Accordingly, the techniques and structures described and illustrated previously herein should be understood to be illustrative and exemplary, and not necessarily limiting upon the scope.

What is claimed is:

1. A particle, comprising:
cellulose acetate and a cargo agent, wherein the cargo agent is non-covalently bonded to the cellulose acetate.

2. The particle of claim 1, in which the cargo agent is fluorescent.

3. The particle of claim 2, wherein the particle exhibits fluorescent ultrabrightness.

4. The particle of claim 2, wherein the particle exhibits fluorescence in the near infrared part of the spectrum.

5. The particle of claim 4, wherein the cargo agent is IR813, IR143, Indocyanine Green (ICG), Methylene Blue, or a combination thereof.

6. The particle of claim 1, wherein the cargo agent is a hydrophobic drug.

7. The particle of claim 1, further comprising a general polymer physically bonded to the cellulose acetate.

8. The particle of claim 7, wherein the general polymer is present on the particle surface, thereby functionalizing the particle surface.

9. The particle of claim 7, wherein the general polymer is an ionic surfactant, a non-ionic surfactant, or a charged polyelectrolyte.

10. The particle of claim 1, wherein the cargo agent dictates a core crystallinity of said particle.

11. The particle of claim 1, wherein the particle has a spherical shape.

12. The particle of claim 1, wherein the particle has a rod shape.

13. The particle of claim 1, wherein the particle has a size of 30-500 nm.

14. A method for making the particle of claim 1, comprising:
dissolving a cellulose acetate in an organic solvent,
dissolving a molecular guest in said organic solvent,
adding said cellulose acetate and said molecular guest in said organic solvent to a miscible non-solvent, said adding being a dropwise adding coupled with stirring, removing said solvent, and
precipitating one or more particles, each particle comprising said cellulose acetate and said molecular guest, wherein said precipitating forms non-covalent bonds between said cellulose acetate and said molecular guest.

15. The method of claim 14, wherein said solvent is removed via dialysis.

16. The method of claim 14, wherein the molecular guest is a fluorescent dye.

17. The method of claim 14, wherein a nature of the organic solvent determines at least one of: particle size, particle surface chemistry, and particle core crystallinity.

18. The method of claim 14, wherein a nature of the molecular guest determines at least one of: particle size, particle shape, particle surface chemistry, and particle core crystallinity,

19. The method of claim 14, further comprising the step of adding a general polymer.

20. The method of, claim 19, wherein the general polymer is present on the particle's surface, thereby functionalizing the particle.

21. The method of claim 19, wherein the general polymer is an ionic surfactant, a non-ionic surfactant, or a charged polyelectrolyte.

22. The method of claim 19, wherein the general polymer determines at least one of: particle size, particle surface chemistry, and particle core crystallinity.
